(19) United States
(12) Patent Application Publication
(10) Pub. No.: US 2006/0270605 A1
(43) Pub. Date: Nov. 30, 2006

(54) Compositions and methods for inhibiting cell growth and modulating gene expression

(75) Inventor: Denise Perry Simmons, Florham Park, NJ (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6780, US

(73) Assignee: Government of the U.S.A., represented by the Secretary, Department of Health and Human Services, Rockville, MD (US)

(21) Appl. No.: 11/497,627

(22) Filed: Aug. 2, 2006

Related U.S. Application Data
(62) Division of application No. 10/513,114, filed on Dec. 8, 2004.
(60) Provisional application No. 60/377,827, filed on May 3, 2002.

(51) Int. Cl. A61K 38/10; C07K 7/08

(52) U.S. Cl. 514/15; 530/328

(57) ABSTRACT

The invention provides a method of inhibiting cancerous cell growth comprising contacting a collection of cancerous cells with a cell growth-inhibiting effective amount of a purified or isolated polypeptide comprising SE ID NO: 6 or variant thereof. The variant of SEQ ID NO: 6 comprises one, two, or three conservative or neutral amino acid substitutions, provided that amino acids 1-6 and 10-12 of SEQ ID NO: 6 remain unchanged.

### TECHNICAL FIELD OF THE INVENTION

The present invention relates to a purified or isolated polypeptide consisting essentially of the amino acid sequence NCEHFVTYCRYG (SEQ ID NO: 1), ENCEHFVNELRYG (SEQ ID NO: 2), NCEHFVNELRYG (SEQ ID NO: 3), RNCEHFVAQLRYG (SEQ ID NO: 4), NCEHFVAQLRYG (SEQ ID NO: 5), or NCEHFVTYLRYG (SEQ ID NO: 6), a purified or isolated nucleic acid consisting essentially of a nucleotide sequence encoding an aforementioned polypeptide, related compositions and host cells, a method of inhibiting cell growth, a method of modulating gene expression, and a method of enhancing the immune response-inducing effect of a vaccine.

### BACKGROUND OF THE INVENTION

The American Cancer Society estimates the lifetime risk that an individual will develop cancer is 1 in 2 for men and 1 in 3 for women. The development of cancer, while still not completely understood, can be enhanced as a result of a variety of risk factors. For example, exposure to environmental factors (e.g., tobacco smoke) might trigger modifications in certain genes, thereby initiating cancer development. Alternatively, these genetic modifications may not require an exposure to environmental factors to become abnormal. Indeed, certain mutations (e.g., deletions, substitutions, etc.) can be inherited from generation to generation, thereby imparting an individual with a genetic predisposition to develop cancer.

The desire of cancer research is the identification of a therapy effective on several different types of cancers. Yet, despite extensive research into the disease, effective cancer therapeutics remain elusive for the medical community. Clinicians have realized limited success with the current standard therapies, chemotherapy, radiation therapy, and surgery, inasmuch as each therapy has inherent limitations. Chemotherapy and radiation therapy cause extensive damage to normal, healthy tissue, despite efforts to target such therapy to abnormal tissue (e.g., tumors). Surgery can be effective in removing masses of cancerous cells; however, even the most talented surgeon cannot ensure complete removal of affected tissue, nor are all tumors in an anatomical location amenable to surgical removal. The limitations of existing therapies are reflected in the 60% 5-year relative survival rate for all cancers combined (Cancer Facts & FIGS. 2001, The American Cancer Society, New York, N.Y.).

Systemic toxicity of drugs is one of the most serious problems of cancer chemotherapy and frequently is dose-limiting. The appearance of various classes of multiple drug resistant cancerous cells renders even good drugs ineffective, since they are expelled from the tumor cells (Ling, Cancer Chemother. Pharmacol. 40: Suppl, S3-S8 (1997)). Various strategies have been used to get around one or both of these difficulties, but they still are among the most intractable problems of cancer therapy. Targeting of drugs specifically to tumor cells has been the goal of many studies. Various protein toxins conjugated to monoclonal antibodies directed to specific tumor antigens have shown some promise as drugs (Pastan, Biochim. Biophys. Acta 1333: C1-C6 (1997)), but severe problems, such as the development of neutralizing antibodies (Chen et al., Gene Ther. 2: 116-123 (1995)), have limited the effectiveness of the method.

Another promising approach is to use cellular receptors for growth factors (Kihara et al., Cancer Res. 55: 71-77 (1985); Carpenter, Curr. Opin. Cell Biol. 5: 261-264 (1993); and Lemaristre et al., Breast Cancer Res. Treat. 32: 97-103 (1994)), cytokines (Strom et al., Annu. Rev. Med. 44: 343-353 (1993); and Waldmann et al., Ann. Intern. Med. 116: 148-160 (1992)), or hormones (Roth et al., Anticancer Drug Des. 10: 655-666 (1994); and Rink et al., Proc. Natl. Acad. Sci. 93: 15063-15068 (1996)) as targets to deliver cytotoxic moieties to the receptor-bearing cells. In this approach, the receptor binds to a ligand that is conjugated to a toxic moiety, resulting in receptor-mediated endocytosis, wherein the ligand-toxic moiety conjugate is internalized, along with the receptor, by the targeted cell. Once inside the cell, the conjugate is susceptible to lysosomal proteases that cleave the linkage between the ligand and toxin, resulting in the release of the toxin from the conjugate. Through this approach, the delivery of a drug to specific cell populations can be achieved.

Other efforts to decrease treatment-related toxicities and enhance specific cytotoxicity of cancer treatment have focused on immunotherapy to generate a specific anticancer immunity, metabolic pathways to target specifically cancerous cells, and identification of genes responsible for the malignant transformation of normal cells into cancerous cells. Clinicians also have looked to the delivery of therapeutic nucleic acid sequences as a possible alternative to existing cancer therapies. The local production of therapeutic agents at biologically-significant levels in target sites in vivo, thereby reducing the toxicity to normal tissues, addresses some of the limitations associated with conventional therapy.

Accordingly, there remains a need for a composition suitable for use in treating a variety of cancer types in a patient, as well as a method for delivering the composition to treat cancer. The present invention provides such a composition and method. This and other objects and advantages of the invention, as well as additional inventive features, will be apparent from the description of the invention provided herein.

### BRIEF SUMMARY OF THE INVENTION

The present invention provides a purified or isolated polypeptide consisting essentially of the amino acid sequence NCEHFVTYCRYG (SEQ ID NO: 1), ENCEHFVNELRYG (SEQ ID NO: 2), NCEHFVNELRYG (SEQ ID NO: 3), RNCEHFVAQLRYG (SEQ ID NO: 4), NCEHFVAQLRYG (SEQ ID NO: 5), or NCEHFVTYLRYG (SEQ ID NO: 6), or a variant of any of the foregoing, any one of which is optionally part of a fusion protein or conjugated to an agent that increases the potency and/or specificity of the polypeptide. Also provided is a composition comprising one or more of the aforementioned polypeptides and a carrier, excipient or adjuvant.

Also provided by the present invention is a purified or isolated nucleic acid consisting essentially of a nucleotide sequence encoding the amino acid sequence of SEQ ID NO: 1, SEQ ID NO: 2, SEQ ID NO: 3, SEQ ID NO: 4, SEQ ID NO: 5, SEQ ID NO: 6, or a variant of any of the foregoing, optionally as part of an encoded fusion protein, and optionally in the form of a vector, which is optionally targeted to a membrane receptor. The nucleic acid can have a peptidic backbone when not in the form of a vector. A composition comprising one or more of the aforementioned nucleic acids and a carrier, excipient or adjuvant is also provided.

A host cell comprising an above-described purified or isolated nucleic acid molecule is also provided. The nucleic acid can have a peptidic backbone when not in the form of a vector.

Further provided is a method of inhibiting cancerous cell growth. The method comprises contacting a collection of cancerous cells with a cell growth-inhibiting effective amount of one or more of the following:

(i) at least one above-described purified or isolated polypeptide, which can be the same or different,

(ii) a composition comprising (i) and a carrier, excipient or adjuvant,

(iii) at least one above-described purified or isolated nucleic acid, which can be the same or different, and

(iv) a composition comprising (iii) and a carrier, excipient or adjuvant, whereupon the growth of the collection of cancerous cells is inhibited. The method optionally further comprises separately contacting the collection of cancerous cells with an anti-cancer agent in the same manner or a different manner, surgical removal of the collection of cancerous cells when in vivo, and/or radiation.

Still further provided is a method of modulating gene expression. The method comprises contacting a collection of cells with a gene expression-modulating effective amount of one or more of the following:

(i) at least one above-described purified or isolated nucleic acid, which can be the same or different and which has a peptidic backbone, and

(ii) a composition comprising (i) and a carrier, excipient or adjuvant, whereupon gene expression in the collection of cells is modulated.

Also still further provided is a method of enhancing the immune response-inducing effect of a vaccine. The method comprises adding to the vaccine at least one above-described purified or isolated polypeptide, which can be the same or different, whereupon the immune response-inducing effect of the vaccine is enhanced.

### DETAILED DESCRIPTION OF THE INVENTION

The present invention is predicated on the surprising and unexpected discovery that tumor suppressor gene polypeptides (TSGP) can dramatically inhibit the proliferation of cancerous cells. The TSGP have anti-proliferative, anti-tumor and cytostatic properties. In this regard, the present invention provides a purified or isolated polypeptide consisting essentially of the amino acid sequence NCEHFVTYCRYG (SEQ ID NO: 1), ENCEHFVNELRYG (SEQ ID NO: 2), NCEHFVNELRYG (SEQ ID NO: 3), RNCEHFVAQLRYG (SEQ ID NO: 4), NCEHFVAQLRYG (SEQ ID NO: 5), or NCEHFVTYLRYG (SEQ ID NO: 6). The term “isolated” as used herein means having been removed from its natural environment. The term “purified” as used herein means having been increased in purity, wherein “purity” is a relative term, and not to be construed as absolute purity. In each embodiment provided herein, a letter indicates the standard amino acid designated by that letter. Additionally, in accordance with convention, all amino acid sequences provided herein are given from left to right, such that the first amino acid is amino-terminal and the last amino acid is carboxyl-terminal. The polypeptide preferably comprises an amino end and a carboxyl end. The polypeptide can comprise D-amino acids, L-amino acids or a mixture of D- and L-amino acids. The D-form of the amino acids, however, is particularly preferred, since a polypeptide comprised of D-amino acids is expected to have a greater retention of its biological activity in vivo, given that the D-amino acids are not recognized by naturally occurring proteases.

The polypeptides (and variants thereof as described herein below) can be prepared by any of a number of conventional techniques. The polypeptide can be isolated or purified from a naturally occurring source or from a recombinant source. For instance, in the case of recombinant polypeptides, a DNA fragment encoding a desired peptide can be subcloned into an appropriate vector using well-known molecular genetic techniques (see, e.g., Maniatis et al., Molecular Cloning: A Laboratory Manual, 2nd ed. (Cold Spring Harbor Laboratory, 1989) and other references cited herein under “EXAMPLE”). The fragment can be transcribed and the polypeptide subsequently translated in vitro. Commercially available kits also can be employed (e.g., such as manufactured by Clontech, Palo Alto, Calif.; Amersham Life Sciences, Inc., Arlington Heights, Ill.; InVitrogen, San Diego, Calif., and the like). The polymerase chain reaction optionally can be employed in the manipulation of nucleic acids.

Such polypeptides also can be synthesized using an automated peptide synthesizer in accordance with methods known in the art. Alternately, the polypeptide (including the variant peptides) can be synthesized using standard peptide synthesizing techniques well-known to those of skill in the art (e.g., as summarized in Bodanszky, Principles of Peptide Synthesis, (Springer-Verlag, Heidelberg: 1984)). In particular, the polypeptide can be synthesized using the procedure of solid-phase synthesis (see, e.g., Merrifield, J. Am. Chem. Soc., 85, 2149-54 (1963); Barany et al., Int. J. Peptide Protein Res., 30, 705-739 (1987); and U.S. Pat. No. 5,424,398). If desired, this can be done using an automated peptide synthesizer. Removal of the t-butyloxycarbonyl (t-BOC) or 9-fluorenylmethyloxycarbonyl (Fmoc) amino acid blocking groups and separation of the polypeptide from the resin can be accomplished by, for example, acid treatment at reduced temperature. The polypeptide-containing mixture then can be extracted, for instance, with dimethyl ether, to remove non-peptidic organic compounds, and the synthesized polypeptide can be extracted from the resin powder (e.g., with about 25% w/v acetic acid). Following the synthesis of the polypeptide, further purification (e.g., using high performance liquid chromatography (HPLC)) optionally can be done in order to eliminate any incomplete polypeptides or free amino acids. Amino acid and/or HPLC analysis can be performed on the synthesized polypeptide to validate its identity. For other applications according to the invention, it may be preferable to produce the polypeptide as part of a larger fusion protein, either by chemical conjugation, or through genetic means, such as are known to those skilled in the art.

The polypeptides of SEQ ID NOS: 1-6 are preferred. However, conservative and neutral amino acid substitutions can be introduced into the polypeptides. In this regard, the present invention also provides a purified or isolated variant of an above-described polypeptide. When tumor suppressor activity is desired, NCEHFV and RYG, i.e., amino acids 1-6 and 10-12 of SEQ ID NOS: 1, 3, 5 and 6, and amino acids 2-7 and 11-13 of SEQ ID 2 and 4, should remain unchanged. Desirably, the leucine at amino acid position 9 in SEQ ID NOS: 3, 5 and 6 also should remain unchanged for tumor suppressor activity. The variant comprises one, two or three conservative or neutral amino acid substitutions, provided that amino acids 8 and 9 in SEQ ID NO: 1, amino acids 1, 8 and 9 in SEQ ID NO: 2, amino acids 7 and 8 in SEQ ID NO: 3, amino acids I and 8 in SEQ ID NO: 4, amino acid 7 in SEQ ID NO: 5, and amino acid 7 in SEQ ID NO: 6 remain unchanged. Where amino acid 7 can be altered, preferably amino acid 7 is uncharged when tumor suppressor activity is desired. Where amino acid 8 is altered, it can have other than a positive charge, such as a neutral or negative charge or hydrophobicity, and still maintain tumor suppressor activity. Additionally or alternatively, the variant comprises one, two or three amino acid additions at the N-terminus and/or C-terminus. Preferably, not more than a total of 1, 2 or 3 amino acids are added. Desirably, the variant has activity characteristic of the unaltered polypeptide, optionally to a greater or lesser extent, but not negated.

Alterations of the native amino acid sequence to produce variant polypeptides can be done by a variety of means known to those skilled in the art. For instance, amino acid substitutions can be conveniently introduced into the polypeptides at the time of synthesis. Alternatively, site-specific mutations can be introduced by ligating into an expression vector a synthesized oligonucleotide comprising the modified site. Alternately, oligonucleotide-directed, site-specific mutagenesis procedures can be used, such as disclosed in Walder et al., Gene, 42, 133 (1986); Bauer et al., Gene, 37, 73 (1985); Craik, Biotechniques, 12-19 (January 1995); and U.S. Pat. Nos. 4,518,584 and 4,737,462.

It is within the skill of the ordinary artisan to select synthetic and naturally-occurring amino acids that effect conservative or neutral substitutions for any particular naturally-occurring amino acids. The skilled artisan desirably will consider the context in which any particular amino acid substitution is made, in addition to considering the hydrophobicity or polarity of the side-chain, the general size of the side chain and the pK value of side-chains with acidic or basic character under physiological conditions. For example, lysine, arginine, and histidine are often suitably substituted for each other, and more often arginine and histidine. As is known in the art, this is because all three amino acids have basic side chains, whereas the pK value for the side-chains of lysine and arginine are much closer to each other (about 10 and 12) than to histidine (about 6). Similarly, glycine, alanine, valine, leucine, and isoleucine are often suitably substituted for each other, with the proviso that glycine is frequently not suitably substituted for the other members of the group. This is because each of these amino acids are relatively hydrophobic when incorporated into a polypeptide, but glycine's lack of an α-carbon allows the phi and psi angles of rotation (around the α-carbon) so much conformational freedom that glycinyl residues can trigger changes in conformation or secondary structure that do not often occur when the other amino acids are substituted for each other. Other groups of amino acids frequently suitably substituted for each other include, but are not limited to, the group consisting of glutamic and aspartic acids; the group consisting of phenylalanine, tyrosine and tryptophan; and the group consisting of serine, threonine and, optionally, tyrosine. Additionally, the ordinarily skilled artisan can readily group synthetic amino acids with naturally-occurring amino acids.

If desired, the polypeptides of the invention (including variant polypeptides) can be modified, for instance, by glycosylation, amidation, carboxylation, or phosphorylation, or by the creation of acid addition salts, amides, esters, in particular C-terminal esters, and N-acyl derivatives of the polypeptides of the invention. The polypeptides also can be modified to create polypeptide derivatives by forming covalent or noncovalent complexes with other moieties in accordance with methods known in the art. Covalently-bound complexes can be prepared by linking the chemical moieties to functional groups on the side chains of amino acids comprising the polypeptides, or at the N- or C-terminus. Desirably, such modifications and conjugations do not adversely affect the activity of the polypeptides (and variants thereof). While such modifications and conjugations can have greater or lesser activity, the activity desirably is not negated and is characteristic of the unaltered polypeptide.

Thus, in this regard, the present invention also provides a fusion protein comprising an above-described isolated or purified polypeptide (or variant thereof) and a conjugate comprising an above-described isolated or purified polypeptide (or variant thereof) and an agent that increases the potency and/or specificity of the polypeptide or variant thereof. Examples of such an agent include, but are not limited to, a ligand or an antibody (or fragment thereof) for a cell-surface receptor or molecule, the expression of which is specific to a particular type of cell, in particular a cancerous cell, such as a cell of melanoma, an immunoprotein sequence known to be selectively expressed (e.g., melanoma-associated antigens, such as MART-1/Melan-A (Schutz et al., Cancer Gene Ther. 8: 655-661 (2001)), or overexpressed on melanoma cells (see, e.g., Brown et al., Curr. Oncol. Rep. 3: 344-352 (2001)), an import peptide, a protein transduction domain, an antisense molecule, such as an antisense molecule that is specific for a gene encoding a retinoic acid receptor (RAR) or an over-expressed receptor, a ribozyme to a gene that is expressed only in a particular type of cell or overexpressed in a particular type of cell, e.g., a cancerous cell, such as an antisense oncogene, e.g., c-fos, a homeobox gene, and an RAR gene, e.g., RAR-β, which has aberrant expression in melanoma, an anti-cancer agent, and the like.

Ligands include, for example, a protein or polypeptide ligand, when the target is a cell-surface receptor, a steroid, when the target is a steroid receptor, and the like. Analogs of targeting moieties that retain the ability to bind to a defined target also can be used. In addition, synthetic targeting moieties can be designed, so as to fit a particular epitope on a cell surface, for example. Alternatively, the polypeptide can be encapsulated in a liposome comprising on its surface a targeting moiety, such as a ligand or an antibody or immunologically reactive fragment thereof.

Examples of cancer-specific, cell-surface receptors include erbB-2, erbB-3, erbB-4, IL-2 (lymphoma and leukemia), IL-4 (lymphoma and leukemia), IL-6 (lymphoma and leukemia), MSH (melanoma), transferrin (gliomas), tumor vasculature integrins, and the like. Preferred cancer-specific, cell-surface receptors include erbB-2 and tumor vasculature integrins, such as CD11a, CD11b, CD11c, CD18, CD29, CD51, CD61, CD66d, CD66e, CD106, and CDw145.

The antibody can be a polyclonal antibody or a monoclonal antibody or an immunologically reactive fragment of either of the foregoing. Alternatively, an engineered immunoprotein can be used. Antibodies, immunologically reactive fragments thereof, and immunoprotiens can be generated in accordance with methods known in the art, including those set forth in the references listed herein under “EXAMPLE.”

There are a number of antibodies to cancer-specific, cell-surface molecules and receptors that are known. C46 Ab (Amersham) and 85A12 Ab (Unipath) to carcino-embryonic antigen, H17E2 Ab (ICRF) to placental alkaline phosphatase, NR-LU-10 Ab (NeoRx Corp.) to pan carcinoma, HMFC1 Ab (ICRF) to polymorphic epithelial mucin, W14 Ab to B-human chorionic gonadotropin, RFB4 Ab (Royal Free Hospital) to B-lymphocyte surface antigen, A33 Ab (Genex) to human colon carcinoma, TA-99 Ab (Genex) to human melanoma, antibodies to c-erbB2 (JP 7309780, JP 8176200 and JP 7059588), and the like. ScAbs can be developed, based on such antibodies, using techniques known in the art (see for example, Bind et al., Science 242: 423-426 (1988), and Whitlow et al., Methods 2(2): 97-105 (1991)).

Import peptides and protein transduction domains can be used to improve transport of the polypeptide across the cell membrane, particularly when the cell membrane is selective or demonstrates poor permeability. See, e.g., Park et al. (J. Gen Virol. 83: 1173-1181 (2002)) with respect to protein transduction domains, and Lanford et al. (Cell 15: 5875-5882 (1986)) and Adam et al. (Cell 66: 837-847 (1988)) with respect to nuclear localization sequences. See, also, Morris et al., Nature Biotech 19: 1173-1175 (2001), and Gilon et al., Biopolymers 31: 745-750 (1991).

The antisense molecule preferably is at least about 20 nucleotides in length, and the ribozyme preferably comprises at least about 20 continuous nucleotides complementary to a target sequence on each side of the active site of the ribozyme. The nucleic acid sequence introduced in antisense suppression generally is substantially identical to at least a portion, preferably at least about 20 contiguous nucleotides, of the gene to be targeted, but need not be identical. A vector expressing an antisense molecule can, thus, be designed such that the inhibitory effect applies to other proteins within a family of genes exhibiting homology or substantial homology to the target gene. The introduced sequence also need not be ful-length relative to either of the primary transcription product or fully processed mRNA. Generally, higher homology can be used to compensate for the use of a shorter sequence. Furthermore, the introduced sequence need not have the same intron or exon pattern, and homology of non-coding segments will be equally effective. As an alternative to antisense suppression, interfering RNA can be used to achieve the same effect by a different mechanism of action.

Ribozymes can be designed such that they specifically pair with virtually any target RNA and cleave the phosphodiester backbone at a specific location, thereby functionally inactivating the target RNA. In carrying out this cleavage, the ribozyme is not itself altered and is, thus, capable of recycling and cleaving other molecules, making it a true enzyme. The inclusion of ribozyme sequences with antisense RNAs confers RNA-cleaving activity upon them, thereby increasing the activity of the constructs. The design and use of target RNA-specific ribozymes is described in Haseloff et al., Nature 334: 585-591 (1988). Preferably, the ribozyme comprises at least about 20 contiguous nucleotides complementary to the target sequence on each side of the active site of the ribozyme.

Alternatively, the polypeptide or variant thereof can be conjugated to a reporter group, including, but not limited to a radiolabel, a fluorescent label, such as fluorescein, an enzyme (e.g., that catalyzes a colorimetric or fluorometric reaction), a substrate, a solid matrix, or a carrier (e.g., biotin or avidin). Methods of conjugation are known in the art. In addition, conjugate kits are commercially available. When the polypeptide or variant thereof is labeled with flourescein, such labeling can be done by Sigma Genosys according to their labeling protocol for 5(6)-carboxyfluorescein. In order to preserve the polypeptide's tumor suppressor activity, maintain the ability of the conjugated fluorophore to emit at its optimal wavelength, and maintain the ability of the polypeptide to cross cellular membranes, the fluorophore should be placed at the N-terminus of the polypeptide, which has the requisite free amine, rather than within or at the C-terminus. Detectably labeled polypeptides can be used to determine whether or not cancerous cells can be treated with the polypeptides of the present invention having tumor suppressor activity. Detection of the labeled polypeptide within the nucleus of a cell indicates that the cell is susceptible to treatment with a present inventive polypeptide. Detectably labeled polypeptides also can be used to identify other molecular targets in the development of cancer, such as melanoma.

In view of the foregoing, the present invention also provides a composition comprising (i) an above-described purified or isolated polypeptide (or variant thereof) and (ii) a carrier, excipient or adjuvant. The polypeptide or variant thereof can be optionally part of a fusion protein or conjugated to an agent that increases the potency and/or specificity of the polypeptide or variant as set forth above. The polypeptides can serve as neoadjuvants to decrease margins of primary tumors in surgical settings.

When the polypeptide consists essentially of the amino acid sequence of SEQ ID NO: 4, SEQ ID NO: 5, SEQ ID NO: 6 or a variant of any of the foregoing, the composition can further comprise a retinoid. Retinoids include agents that bind to the retinoic acid receptor, such as 9-cis-retinoic acid, 4-hydroxy-retinoic acid, all trans-retinoic acid, (E)-4-[2-(5,6,7,8-tetrahydro-2-naphthylenyl)-1-propenyl]-benzoic acid, and 3-methyl-(E)4-[2-(5,6,7,8-tetrahydro-2-naphthylenyl)-1 -propenyl]-benzoic acid).

Optionally, the composition further comprises an anti-cancer agent. Examples of anti-cancer agents include those set forth herein below.

Preferably, the carrier is pharmaceutically acceptable. The carrier can be any of those conventionally used and is limited only by chemico-physical considerations, such as solubility and lack of reactivity with the active agent of the present invention, and by the route of administration. It is preferred that the pharmaceutically acceptable carrier be one which is chemically inert to the active agent and one which has no detrimental side effects or toxicity under the conditions of use. The pharmaceutically acceptable carriers described herein, for example, vehicles, adjuvants, excipients, and diluents, are well-known to those ordinarily skilled in the art and are readily available to the public. Typically, the composition, such as a pharmaceutical composition, can comprise a physiological saline solution; dextrose or other saccharide solution; or ethylene, propylene, polyethylene, or other glycol. The TSGP are soluble in water.

Also in view of the foregoing, the present invention provides a purified or isolated nucleic acid consisting essentially of a nucleotide sequence encoding an above-described amino acid sequence (or variant thereof). The terms “purified” and “isolated” have the meaning set forth above. The term “nucleic acid” as used herein means a polymer of DNA or RNA, (i.e., a polynucleotide), which can be single-stranded or double-stranded, synthesized or obtained from natural sources, and which can contain natural, non-natural or altered nucleotides. With respect to the isolated or purified nucleic acid of the present invention, it is preferred that no insertions, deletions, inversions, and/or substitutions are present in the nucleic acid. When tumor suppressor activity is desired, the nucleic acid should encode amino acids 1-6 and 10-12 of SEQ ID NOS: 1, 3, 5 and 6, and amino acids 2-7 and 11-13 of SEQ ID NOS: 2 and 4. Desirably, the nucleic acid also should encode amino acid 9 in SEQ ID NOS: 3, 5 and 6 when tumor suppressor activity is desired. However, it may be suitable in some instances for the isolated or purified nucleic acid to encode one or more conservative and/or neutral amino acid substitutions, provided that amino acids 8 and 9 in SEQ ID NO: 1, amino acids 1, 8 and 9 in SEQ ID NO: 2, amino acids 7 and 8 in SEQ ID NO: 3, amino acids 1 and 8 in SEQ ID NO: 4, and amino acid 7 in SEQ ID NO: 5 remain unchanged. Where amino acid 7 can be altered in one of the present inventive polypeptides and tumor suppressor activity is desired, preferably the nucleic acid encodes an amino acid at position 7 that is uncharged. Where amino acid 8 can be altered in one of the present inventive polypeptides and tumor suppressor activity is desired, the nucleic acid can encode an amino acid at position 8 that has other than a positive charge, such as a neutral or negative charge, or hydrophobicity. In addition to conservative and neutral amino acid substitutions, nucleotides can be added at the 5′ and/or 3′ end of the nucleic acid such that amino acids are added to the N and/or C terminus of the encoded polypeptide. Preferably, not more than up to a total of 1, 2 or 3 amino acids are added. Desirably, the encoded variant has activity characteristic of the unaltered polypeptide, optionally to a greater or lesser extent, but not negated. A variety of techniques used to synthesize the oligonucleotides of the present invention are known in the art. See, for example, Lemaitre et al., Proceedings of the National Academy of the Sciences 84: 648-652 (1987).

The nucleic acid can have a peptidic backbone, preferably when it is not part of a vector. Such nucleic acids are referred to as peptidic nucleic acids or PNAs, functional properties of which optimize modulation of gene expression include length, target sequence and G/C content (see, e.g., Doyle et al., Biochem 40: 53-64 (2001)). PNAs can be obtained from Applied Systems (Framingham, Mass.), or synthesized using the t-BOC strategy of solid phase synthesis (see, e.g., Langel et al., Int. J. Pept. Pro Res. 39: 516-522 (1992)) or another synthesis method (see, e.g., Christensen et al., J. Pept. Sci. 3:175 (1995); Haaima et al., Angew. Chem., Int. Ed. Engl. 35: 1939 (1996); and Paschl et al., Tetrahedron Lett. 39: 4707 (1998)). Parity of the PNA can be demonstrated by HPLC. The molecular mass can be determined by mass spectrometry. PNAs for the polypeptides of SEQ ID NOS: 1-6 and variants thereof will range from 9 to about 36 bases. PNA also can be labeled with a detectably labeled probe, such as those known in the art, e.g., biotin, FITC and anthracene, and used as a probe to identify or assess the mechanism or molecular interaction of TSGPs with DNA.

In view of the above, the present invention also provides a vector comprising an above-described isolated or purified nucleic acid molecule, optionally as part of an encoded fusion protein. The vector can be targeted to a membrane receptor if so desired. A nucleic acid molecule as described above can be cloned into any suitable vector and can be used to transform or transfect any suitable host. The selection of vectors and methods to construct them are commonly known to persons of ordinary skill in the art and are described in general technical references (see, in general, “Recombinant DNA Part D,” Methods in Enzymology, Vol. 153, Wu and Grossman, eds., Academic Press (1 987) and the references cited herein under “EXAMPLE”). Desirably, the vector comprises regulatory sequences, such as transcription and translation initiation and termination codons, which are specific to the type of host (e.g., bacterium, fungus, plant or animal) into which the vector is to be introduced, as appropriate and taking into consideration whether the vector is DNA or RNA. Preferably, the vector comprises regulatory sequences that are specific to the genus of the host. Most preferably, the vector comprises regulatory sequences that are specific to the species of the host.

Constructs of vectors, which are circular or linear, can be prepared to contain an entire nucleic acid as described above or a portion thereof ligated to a replication system functional in a prokaryotic or eukaryotic host cell. Replication systems can be derived from ColE1, 2 mμ plasmid, λ, SV40, bovine papilloma virus, and the like.

In addition to the replication system and the inserted nucleic acid, the construct can include one or more marker genes, which allow for selection of transformed or transfected hosts. Marker genes include biocide resistance, e.g., resistance to antibiotics, heavy metals, etc., complementation in an auxotrophic host to provide prototrophy, and the like.

One of ordinary skill in the art will appreciate that any of a number of vectors known in the art are suitable for use in the invention. Suitable vectors include those designed for propagation and expansion or for expression or both. Examples of suitable vectors include, for instance, plasmids, plasmid-liposome complexes, and viral vectors, e.g., parvoviral-based vectors (i.e., adeno-associated virus (AAV)-based vectors), retroviral vectors, herpes simplex virus (HSV)-based vectors, and adenovirus-based vectors. Any of these expression constructs can be prepared using standard recombinant DNA techniques described in, e.g., Sambrook et al., Molecular Cloning: A Laboratory Manual, 2^{nd} edition, Cold Spring Harbor Laboratory Press, Cold Spring Harbor, N.Y. (1989); and Ausubel et al., Current Protocols in Molecular Biology, Greene Publishing Associates and John Wiley & Sons, New York, N.Y. (1994). Examples of cloning vectors include the pUC series, the pBluescript series (Stratagene, LaJolla, Calif.), the pET series (Novagen, Madison, Wis.), the pGEX series (Pharmacia Biotech, Uppsala, Sweden), and the pEX series (Clonetech, Palo Alto, Calif.). Bacteriophage vectors, such as λGT10, λGT11, λZapII (Stratagene), λ EMBL4, and λ NM1149, also can be used. Examples of plant expression vectors include pBI101, pBI101.2, pBI101.3, pBI121 and pBIN19 (Clonetech, Palo Alto, Calif.). Examples of animal expression vectors include pEUK-C1, pMAM and pMAMneo (Clonetech).

An expression vector can comprise a native or nonnative promoter operably linked to an isolated or purified nucleic acid as described above. The selection of promoters, e.g., strong, weak, inducible, tissue-specific and developmental-specific, is within the skill in the art. Similarly, the combining of a nucleic acid molecule as described above with a promoter is also within the skill in the art.

Optionally, the isolated or purified nucleic acid molecule, upon linkage with another nucleic acid molecule, can encode a fusion protein. The generation of fusion proteins is within the ordinary skill in the art (see, e.g., references cited under “Example”) and can involve the use of restriction enzyme or recombinational cloning techniques (see, e.g., Gateway™ (Invitrogen, Carlsbad, Calif.)). See, also, U.S. Pat. No. 5,314,995.

A targeting moiety also can be used in the contact of a cell with an above-described isolated or purified nucleic acid or variant thereof. In this regard, any molecule that can be linked with the nucleic acid directly or indirectly, such as through a suitable delivery vehicle, such that the targeting moiety binds to a membrane receptor, such as a cell-surface receptor or nuclear membrane receptor, or other molecule, can be used. The targeting moiety can bind to a cell through a receptor, a substrate, an antigenic determinant or another binding site on the surface of the cell. Examples of a targeting moiety include an antibody (i.e., a polyclonal or a monoclonal antibody), an immunologically reactive fragment of an antibody, an engineered immunoprotein and the like, a protein (target is receptor, as substrate, or regulatory site on DNA or RNA), a polypeptide (target is receptor), a peptide (target is receptor), a steroid (target is steroid receptor), and the like. Analogs of targeting moieties that retain the ability to bind to a defined target also can be used. In addition, synthetic targeting moieties can be designed, such as to fit a particular epitope. Alternatively, the nucleic acid can be encapsulated in a liposome comprising on its surface the targeting moiety. Examples of cell-surface receptors, ligands, and antibodies are set forth above.

The targeting moiety includes any linking group that can be used to join a targeting moiety to, in the context of the present invention, an above-described nucleic acid molecule. It will be evident to one skilled in the art that a variety of linking groups, including bifunctional reagents, can be used. The targeting moiety can be linked to the nucleic acid by covalent or non-covalent bonding. If bonding is non-covalent, the conjugation can be through hydrogen bonding, ionic bonding, hydrophobic or van der Waals interactions, or any other appropriate type of binding.

Thus, the present invention further provides a composition comprising (i) one or more of an above-described purified or isolated nucleic acid or variant thereof, optionally as part of an encoded fusion protein, and (ii) a carrier, excipient or adjuvant. The nucleic acid is optionally in the form of a vector, which is optionally targeted to a membrane receptor. When the nucleic acid is not in the form of a vector, the nucleic acid optionally can have a peptidic backbone. When the nucleic acid encodes the amino acid of SEQ ID NO: 4, the amino acid of SEQ ID NO: 5, the amino acid of SEQ ID NO: 6, or a variant of any one or more of the foregoing, the composition can further comprise a retinoid. The composition can further comprise an anti-cancer agent, examples of which are set forth herein below. Suitable carriers, excipients and adjuvants are known in the art and described herein. Suitable compositions are as set forth herein.

A host cell comprising an above-described purified or isolated nucleic acid or variant thereof, optionally as part of an encoded fusion protein, and optionally in the form of a vector, which is optionally targeted to a membrane receptor, is also provided. When the nucleic acid or variant thereof is not in the form of a vector, the nucleic acid optionally can have a peptidic backbone.

Examples of host cells include, but are not limited to, a human cell, a human cell line, E. coli, B. subtilis, P. aerugenosa, S. cerevisiae, and N. crassa E. coli, in particular E. coli TB-1, TG-2, DH5a, XL-Blue MRF′ (Stratagene), SA2821 and Y1090 are preferred hosts.

A method of inhibiting cancerous cell growth is also provided. The method comprises contacting a collection of cancerous cells with a cell growth-inhibiting effective amount of one or more of the following:

(i) at least one purified or isolated polypeptide or variant thereof as described herein, which can be the same or different, and which can be optionally targeted to an agent that increases the potency and/or specificity of the polypeptide or variant thereof,

(ii) a composition comprising (i) and a carrier, excipient or adjuvant, and, optionally, an anti-cancer agent, and, when the polypeptide consists essentially of the amino acid sequence of SEQ ID NO: 4, SEQ ID NO: 5 or SEQ ID NO: 6, optionally, a retinoid,

(iii) at least one purified or isolated nucleic acid or variant thereof as described herein, which can be the same or different, optionally in the form of a vector, which is optionally targeted to a cell-surface receptor on the collection of cells, with the proviso that, when the purified or isolated nucleic acid is not in the form of a vector, the purified or isolated nucleic acid can have a peptidic backbone, and

(iv) a composition comprising (iii) and a carrier, excipient or adjuvant, and, optionally, an anti-cancer agent, and when the nucleic acid encodes the amino acid of SEQ ID NO: 4, SEQ ID NO: 5 or SEQ ID NO: 6, optionally retinoic acid. “Membrane receptor” is used generically to refer to a cell-surface receptor, cell-surface molecule, nuclear membrane receptor, and nuclear membrane molecule. The method can further comprise separately contacting the collection of cells with an anti-cancer agent, which can be the same or different, in the same manner or a different manner, surgical removal of the collection of cancer cells when in vivo, and/or radiation, and the like.

A “cell growth-inhibiting effective amount” is an amount sufficient to inhibit the growth. i.e., increase in cellular mass or cellular replication or proliferation, of the collection of cells, such as cancerous cells, to any degree or to inhibit the onset of cancer. Preferably, the collection of cells is in vivo. A particularly preferred collection of cells in vivo is melanoma.

Preferably, the collection of cells is in a mammal. For purposes of the present invention, mammals include, but are not limited to, the order Rodentia, such as mice, and the order Logomorpha, such as rabbits, the order Carnivora, including Felines (cats) and Canines (dogs), the order Artiodactyla, including Bovines (cows) and Suines (pigs), the order Perssodactyla, including Equines (horses), the order Primate, Ceboid, or Simoid (monkeys), or the order Anthropoids (humans and apes). An especially preferred mammal is the human.

Any suitable method can be used to contact the collection of cells and will depend on whether the cells are in vitro or in vivo. If the collection of cells is in vivo, the method used to contact the collection of cells will also depend on whether the collection of cells is on the surface of the body or internal to the body. Suitable routes of in vivo administration are known in the art and include, oral, topical, epidermal, intradermal, transdermal, systemic, intravenous, perenteral, intraperitoneal, and the like. One skilled in the art will appreciate that suitable methods of administering the active agents of the present invention or composition thereof to an animal, e.g., a mammal such as a human, are known, and, although more than one route can be used to administer a particular composition, a particular route can provide a more immediate and more effective reaction than another route.

Desirably, the active agent is administered in the form of a pharmaceutically acceptable composition (see, e.g., Remington's Pharmaceutical Sciences, 17th ed., (Mack Publishing Company, Philadelphia, Pa.: 1985), and Langer, Science, 249, 1527-1533 (1990)) suitable for topical administration. Such compositions are known in the art. A formulation suitable for topical application can be in the form of creams, ointments, or lotions in which the inhibitor can be mixed with conventional oleoginous or emulsifying excipients.

Formulations suitable for oral administration can consist of (a) liquid solutions, such as an effective amount of the conjugate dissolved in diluents, such as water or saline, (b) capsules, sachets or tablets, each containing a predetermined amount of the active ingredient, as solids or granules, (c) suspensions in an appropriate liquid, and (d) suitable emulsions.

Tablet forms can include one or more of lactose, mannitol, cornstarch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions gels, and the like containing, in addition to the active ingredient, such carriers as are known in the art.

Formulations suitable for parenteral administration include aqueous and non-aqueous solutions, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The dose administered to an animal, particularly a human, in the context of the present invention should be sufficient to effect the desired response in the animal over a reasonable time frame. The dose will be determined by the strength of the particular polypeptide, nucleic acid, or composition and the condition of the animal (e.g., human), as well as the body weight of the animal (e.g., human) to be treated. The size of the dose also will be determined by the existence, nature, and extent of any adverse side effects that might accompany the administration of a particular polypeptide, nucleic acid, or composition. A suitable dosage for internal administration is 0.01 to 100 mg/kg per day. A preferred dosage is 0.01 to 35 mg/kg per day. A more preferred dosage is 0.05 to 5 mg/kg per day. A suitable concentration of the conjugate in pharmaceutical compositions for topical administration is 0.05 to 15% (by weight). A preferred concentration is from 0.02 to 5%. A more preferred concentration is from 0.1 to 3%. The TSGP is effective at 2 μM in vitro over a 48-hour period and at 5 μg/kg per injection in vivo. As few as three injections can result in inhibition of tumor growth. The size of a tumor can be maintained with daily injections. Ultimately, the attending physician will decide the dosage and the amount of conjugate of the present invention with which to treat each individual patient, taking into consideration a variety of factors, such as age, body weight, general health, diet, sex, polypeptide, nucleic acid, or composition to be administered, route of administration, whether individual cells, a tissue, an organ or an organism is being contacted, the particular site being treated, and degree of inhibition needed.

A retinoid is preferably administered topically or systemically, depending on the type of cancer. For example, if the cancer is melanoma, desirably, the retinoid is administered topically. If the cancer is internal to the body, the retinoid is desirably administered systemically.

The method can be used in combination with other known treatment methods, such as radiation, surgery, or the administration of other active agents, such as adjuvants or other anti-cancer agents and their prodrugs. Examples of cytotoxic agents and their prodrugs include genistein, okadaic acid, 1-β-D-arabinofuranosyl-cytosine, arabinofuranosyl-5-aza-cytosine, cisplatin, carboplatin, actinomycin D, asparaginase, bis-chloro-ethyl-nitroso-urea, bleomycin, chlorambucil, cyclohexyl-chloro-ethyl-nitroso-urea, cytosine arabinoside, daunomycin, etoposide, hydroxyurea, melphalan, mercaptopurine, mitomycin C, nitrogen mustard, procarbazine, teniposide, thioguanine, thiotepa, vincristine, 5-fluorouracil, 5-fluorocytosine, adriamycin, cyclophosphamide, methotrexate, vinblastine, doxorubicin, leucovorin, taxol, anti-estrogen agents such as tamoxifen, intracellular antibodies against oncogenes, the flavonol quercetin, Guan-mu-tong extract, retinoids such as fenretinide, nontoxid retinoid analogues such as N-(4-hydroxyphenyl)-retinamide (HPR), and monoterpenes such as limonene, perillyl alcohol and sobrerol. The anti-cancer agent can be a chemotherapeutic agent, e.g., a polyamine or an analogue thereof. Examples of therapeutic polyamines include those set forth in U.S. Pat. Nos. 5,880,161, 5,541,230 and 5,962,533; Saab et al., J. Med. Chem. 36: 2998-3004 (1993); Bergeron et al., J. Med. Chem. 37(21): 3464-3476 (1994); Casero et al., Cancer Chemother. Pharmacol 36: 69-74 (1995); Bernacki et al., Clin. Cancer Res. 1: 847-857 (1995); Bergeron et al., J. Med. Chem. 40: 1475-1494 (1997); Gabrielson et al., Clinical Cancer Res. 5: 1638-1641 (1999); and Bergeron et al., J. Med. Chem. 43: 224-235 (2000), which can be administered alone or in combination with other active agents, such as anti-cancer agents, e.g., cis-diaminedichloroplatinum (II) and 1,3-bis(2-chloroethyl)-1-nitrosourea.

Other actions that can be taken include the administration of tumor-infiltrating lymphocytes that express cytokines, RGD-containing peptides and proteins, which can be administered following surgery, or lipophilic drug-containing liposomes to which are covalently conjugated monoclonal antibodies for targeting to cancer cells, adherence to a low fat diet, moderate physical exercise and hormonal modulation. For prostate cancer, anti-testosterone agents can be used as well as an inhibitor of cellular proliferation produced by prostatic stromal cells and C-CAM, an epithelial cell adhesion molecule.

When the collection of cancerous cells is in the form of a tumor, preferred routes of administration include intratumoral and peritumoral routes of administration. A preferred manner of administering a separate anti-cancer agent is by targeting to a cancer cell. In this regard, examples of cancer-specific, cell-surface molecules are as set forth above.

Generally, when an above-described polypeptide is administered to an animal, such as a mammal, in particular a human, it is preferable that the polypeptide is administered in a dose of from about 1 to about 1,000 micrograms of the polypeptide per kg of the body weight of the host per day when given parenterally. However, this dosage range is merely exemplary, and higher or lower doses may be chosen in appropriate circumstances. For instance, the actual dose and schedule can vary depending on whether the composition is administered in combination with other pharmaceutical compositions, or depending on interindividual differences in pharmacokinetics, drug disposition, and metabolism. One skilled in the art easily can make any necessary adjustments in accordance with the necessities of the particular situation.

If desired, the half-life of the polypeptide can be increased by conjugation to soluble macromolecules, such as polysaccharides, or synthetic polymers, such as polyethylene glycol, as described, for instance, in U.S. Pat. Nos. 5,116,964, 5,336,603, and 5,428,130. Alternately, the polypeptides can be “protected” in vesicles composed of substances such as proteins, lipids (for example, liposomes), carbohydrates, or synthetic polymers. If liposomes are employed, liposome delivery can be carried out as described in U.S. Pat. No. 5,468,481, or using liposomes having increased transfer capacity and/or reduced toxicity in vivo (see, e.g., international patent application WO 95/21259 and the references cited therein). Furthermore, polypeptides can be administered in conjunction with adenovirus (preferably replication-deficient adenovirus) to allow the intracellular uptake of the polypeptides by adenoviral-mediated uptake of bystander molecules (e.g., as described in international patent application WO 95/21259). Similarly, a fusion of an above-described polypeptide and an antibody (or an antigenically reactive fragment thereof) that recognizes a cell surface antigen; etc. as described can be employed to deliver the resultant fusion protein to a specific target cell or tissue (e.g., as described in U.S. Pat. No. 5,314,995).

Those of ordinary skill in the art can easily make a determination of the amount of an above-described isolated and purified nucleic acid molecule to be administered to an animal, such as a mammal, in particular a human. The dosage will depend upon the particular method of administration, including any vector or promoter utilized. For purposes of considering the dose in terms of particle units (pu), also referred to as viral particles, it can be assumed that there are 100 particles/pfu (e.g., 1×10¹² pfu is equivalent to 1×10¹⁴ pu). An amount of recombinant virus, recombinant DNA vector or RNA genome sufficient to achieve a tissue concentration of about 10² to about 10¹² particles per ml is preferred, especially of about 10⁶ to about 10¹⁰ particles per ml. In certain applications, multiple daily doses are preferred. Moreover, the number of doses will vary depending on the means of delivery and the particular recombinant virus, recombinant DNA vector or RNA genome administered.

A method of modulating gene expression is also provided by the present invention. By “modulating” is meant an increase or decrease in gene expression as desired. The method comprises contacting a collection of cells with a gene expression-modulating effective amount of one or more of the above-described nucleic acids (or variants thereof), which can be the same or different, optionally in the form of a composition further comprising a carrier, excipient or adjuvant, provided that the purified or isolated nucleic acid has a peptidic backbone. By “gene expression-modulating effective amount” is mean an amount of the nucleic acid (or variant thereof) or composition comprising same that can effectively increase or decrease the expression of a target gene as desired. When the composition comprises a nucleic acid encoding SEQ ID NO: 4, SEQ ID NO: 5, SEQ ID NO: 6 and/or a variant of any of the foregoing, the composition preferably further comprises a retinoid as described herein. Preferably, the collection of cells is in vivo, such as in a mammal. Preferably, the cells are cancerous cells, in particular cells of melanoma, in which case the gene is RAR β or high mobility group (HMG) I (Y), both of which have a retinoic acid response element in their promoter regions, and the expression of the gene is upregulated. Methods of contacting a collection of cells and suitable compositions for use in the method are as described herein. While not wishing to be bound to any particular theory, it is believed that the present inventive polypeptides can modulate gene expression by binding to a retinoic acid response element, e.g., DR5, in the promoter region of a gene.

Also provided is a method of enhancing the immune response-inducing effect of a vaccine. The method comprises adding to the vaccine at least one above-described purified or isolated polypeptide (or variant thereof), which can be the same or different, optionally as part of a fusion protein or conjugated to an agent that increases the potency and/or specificity of the polypeptide or variant thereof.

### EXAMPLES

The following references, to the extent that they provide exemplary procedural or other details supplementary to those set forth herein, are specifically incorporated herein by reference:
Birren et al., Genome Analysis: A Laboratory Manual Series, Volume 1, Analyzing DNA, Cold Spring Harbor Laboratory Press, Cold Spring Harbor, N.Y. (1997),
Birren et al., Genome Analysis: A Laboratory Manual Series, Volume 2, Detecting Genes, Cold Spring Harbor Laboratory Press, Cold Spring Harbor, N.Y. (1998),
Birren et al., Genome Analysis: A Laboratory Manual Series, Volume 3, Cloning Systems, Cold Spring Harbor Laboratory Press, Cold Spring Harbor, N.Y. (1999),
Birren et al., Genome Analysis: A Laboratory Manual Series, Volume 4, Mapping Genomes, Cold Spring Harbor Laboratory Press, Cold Spring Harbor, N.Y. (1999),
Harlow et al., Antibodies: A Laboratory Manual, Cold Spring Harbor Laboratory Press, Cold Spring Harbor, N.Y. (1988),
Harlow et al., Using Antibodies: A Laboratory Manual, Cold Spring Harbor Laboratory Press, Cold Spring Harbor, N.Y. (1999),
Hoffman, Cancer and the Search for Selective Biochemical Inhibitors, CRC Press (1999),
Pratt, The Anticancer Drugs, 2nd edition, Oxford University Press, NY (1994), and
Sambrook et al., Molecular Cloning: A Laboratory Manual, 2nd edition, Cold Spring Harbor Laboratory Press, Cold Spring Harbor, N.Y. (1989).

The following example serves to illustrate the present invention and should not be construed as in any way limiting its scope.

TSGPs were designed based on the published 12-amino acid sequence homology of human lecithin retinol acyltransferase (LRAT), human Rev (H-Rev), and human tazarotene-induced gene 3 (H-TIG3). Lyophilized TSGPs were synthesized by SIGMA-GENOSYS (The Woodlands, Tex.) and solubilized in sterile ultra pure water (KD Medical, Columbia, Md.) to achieve a 10 mM stock. Aliquots were prepared for one-time use and stored at −80° C.

Melanoma cells were purchased from the American Type Culture Collection (ATCC, Rockville, Md.) and maintained as specified by the ATCC. Specifically, the melanoma cells were Hs939.T, designated as a malignant melanoma cell line and the matched tumor pairs [688(A).T, 688(B).T] and [WM-115, WM-266-4]. Each pair was obtained from a single patient and consists of the primary site tumor cells and cells of a tumor that metastasized from the primary site. Normal human primary epidermal melanocytes (HFSC/2) were obtained from the Yale Skin Diseases Research Center (New Haven, Conn.) and, upon arrival, were maintained in melanocyte growth medium from Clonetics (San Diego, Calif.). The purity of the normal human primary epidermal melanocyte cultures was achieved by growth media restriction of contaminating fibroblasts and keratinocytes. All cell types were identified as adult tissue in origin.

Cultures were seeded, in duplicate, in 6-well dishes to achieve 60-70 % confluence overnight. The next day, 10 mM stocks of TSGPs were diluted in sterile ultra pure water for a working solution of 10 μM. Monolayers were either mock-treated, untreated, or treated with TSGP to a final concentration of 1 μM. Fresh dilutions of TSGPs were prepared daily and added at time zero and 24 hrs. Untreated cells were harvested at 24 and 48 hrs and treated cells were harvested at 48 hrs by trypsinization and resuspensed in growth media and a 1:1 solution of 0.1 % trypan blue. The monolayer from each well and its respective medium were counted for live and dead cells using a hemacytometer. Expected cell proliferation was assessed by comparing the cell number at 24 and 48 hrs in the untreated group.

Daily treatment over 48 hrs of cultures of human melanoma cells, Hs 939.T, with 1 μM TSGP:H-TIG3 decreased the number of proliferating cells by 65.6 %, while treatment with 1 μM TSGP:H-Rev107 reduced the number of proliferating cells by 44%. Importantly, daily treatment of cultures of proliferating normal human melanocytes with 1 μM TSGPs had no significant effect on cell proliferation.

When cultures of Hs 939.T were treated daily over a 48 hour-period with 1 μM TSGP:HTIG3 and 1 μM 9-cis retinoic acid (RA), the observed decrease in cell number with TSGP:H-TIG3 alone was not enhanced. Combination treatment with TSGP:H-Rev107 was enhanced so that the reduction in cell number was increased from 44% to 57%. Either TSGP alone or in combination with 9-cis RA was more effective than 9-cis RA alone.

When 1 μM TSGPs were incubated with cultures of primary site tumor cells, Hs 688(A).T and WM-115, cell proliferation was decreased 45% and 57%, respectively, within 48 hours. However, cultures of metastasized tumors from the primary tumors 688(B).T and WM-266-4 resulted in a 10% and 25%, respectively, reduction in cell number.

Parallel cultures of treated and untreated cells were harvested by lysing the monolayers with SDS lysis buffer and probed with antibodies to HMG I (Y) and RAR β (Santa Cruz Biotechnology, Santa Cruz, Calif.). Protein (20 μg) was loaded on a 4-12% Bis-Tris SDS-PAGE (Novex, Carlsbad, Calif.; Invitrogen, Carlsbad, Calif.) reducing gel, separated by electrophoresis, and transferred onto a 0.2 μm polyvinyldiene difluoride membrane (PVDF). Blots were probed with antibody to HMG I (Y) and RAR β. Signal detection was performed using polyclonal goat anti-mouse conjugated to horseradish peroxidase (Biorad, Hercules, Calif.) in combination with the West Pico SuperSignal chemiluminescent kit (Pierce, Rockford, Ill.). Actin was detected with a polyclonal rabbit anti-actin (Chemicon International, Temecula, Calif.). Specific signal was quantified by ImageQuant Analysis (Molecular Dynamics, Sunnyvale, Calif.). Treatment groups were normalized against densitometry units for untreated, normal human proliferating melanocytes, set at a value of 1.

Untreated melanoma cells, Hs 939.T, were downregulated for RAR β and HMG I (Y) relative to proliferating normal human melanocytes (HFSC/2). When melanoma cultures of Hs 939.T were treated with the TSGP:H-TIG3, the protein expression levels of HMG I (Y) and RAR β were returned to normal, while treatment with TSGP:H-Rev107 resulted in a near normal return of each transcription factor level.

WM-115 cells from benign cutaneous melanoma (ATCC catalog no. 1675) were grown in normal growth medium and then harvested, washed, and suspended in phosphate-buffered saline. Five six-week old Balb/c nu/nu female mice were injected subcutaneously with 5-6×10⁵ cells/animal. Mice were checked weekly for weight and tumor growth. In order to determine antitumor activity, tumors were allowed to grow at least 20 mm³, at which point intratumoral injections of peptide (SEQ ID NO: 3 or SEQ ID NO: 4) at 5 μg/kg animal were given three times over a two-week period. Measurements were taken every other day after peptide treatment until the animal was terminated. To assess whether the peptides were able to maintain the tumor size, tumors were treated as described above, then tumors were allowed to re-establish growth. Intratumoral injections were given and tumor measurements taken daily, over a 5 day period. Overall, a 3 to 13 fold reduction in tumor size was observed upon treatment. The polypeptides not only inhibited tumor growth after three injections, but maintained the size of the tumor with daily injections.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-43. (canceled)

44. A method of inhibiting cancerous cell growth, which method comprises contacting a collection of cancerous cells with a cell growth-inhibiting effective amount of a purified or isolated polypeptide comprising SEQ ID NO: 6 or variant thereof comprising one, two, or three conservative or neutral amino acid substitutions, provided that amino acids 1-6 and 10-12 of SEQ ID NO: 6 remain unchanged.

45. The method of claim 44, wherein the amino acid at position 9 of SEQ ID NO: 6 is unchanged.

46. The method of claim 44, wherein the cancerous cells are in vivo.

47. The method of claim 46, wherein the cancerous cells are in a mammal.

47. The method of claim 44, wherein the cancerous cells are melanoma cells.

48. The method of claim 44, wherein the polypeptide comprises SEQ ID NO: 2.

49. The method of claim 44, wherein the polypeptide comprises SEQ ID NO: 3.

50. The method of claim 44, wherein the polypeptide comprises SEQ ID NO: 4.

51. The method of claim 44, wherein the polypeptide comprises SEQ ID NO: 5.


SEQUENCE LISTING (6 sequences)

SEQ ID NO: 1 - protein, 12 aa, Artificial Sequence
  Synthetic
NCEHFVTYCRYG

SEQ ID NO: 2 - protein, 13 aa, Artificial Sequence
  Synthetic
ENCEHFVNELRYG

SEQ ID NO: 3 - protein, 12 aa, Artificial Sequence
  Synthetic
NCEHFVNELRYG

SEQ ID NO: 4 - protein, 13 aa, Artificial Sequence
  Synthetic
RNCEHFVAQLRYG

SEQ ID NO: 5 - protein, 12 aa, Artificial Sequence
  Synthetic
NCEHFVAQLRYG

SEQ ID NO: 6 - protein, 12 aa, Artificial
  Synthetic
NCEHFVTYLRYG